(19) United States
(12) Patent Application Publication
(10) Pub. No.: US 2004/0107512 A1
(43) Pub. Date: Jun. 10, 2004

(54) Use of thiophenes or bisthiophenes in compositions for treating human keratin fibers, compositions comprising them and processes

(76) Inventors: Henri Samain, Bievres (FR); Gregory Plos, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/355,228

(22) Filed: Jan. 31, 2003

Related U.S. Application Data
(60) Provisional application No. 60/353,992, filed on Feb. 5, 2002.

(30) Foreign Application Priority Data
Jan. 31, 2002 (FR) 02 01145

(51) Int. Cl. A61K 7/13

(52) U.S. Cl. 8/405

(57) ABSTRACT

The invention relates to the use of thiophenes and bisthiophenes as agents making it possible to impart a volume effect and/or a visual effect to human keratin fibers and more particularly the hair.

The invention also relates to the use of thiophenes or bisthiophenes capable of generating via oxidative polymerization a polymer that absorbs in the visible spectrum, as an oxidation dye precursor, in a composition for dyeing human keratin fibers.

The invention also relates to a composition comprising a thiophene or bisthiophene, and to processes using these compounds.

The invention relates to the use of thiophenes and bisthiophenes as agents imparting a volume effect and/or a visual effect to human keratin fibers, in particular the hair, in a composition for treating such fibers. A subject of the invention is also the use of these compounds in compositions for dyeing human keratin fibers. The invention also relates to the treating composition containing them and to processes using it.

There is a need to reinforce human keratin fibers, and more particularly the hair, which have been damaged by natural external agents (for example light) and by chemical or physical cosmetic agents (permanent-waving, dyeing, shampooing, blow-drying, etc.).

With this aim, it has already been proposed to use unsaturated monomers of ethylenic or vinyl type which polymerize inside the fiber.

Unfortunately, such monomers have toxicological characteristics that are poorly suited to cosmetic requirements and their implementation requires conditions that are often extreme. Moreover, their intrinsic instability frequently results in formulation constraints that are not readily acceptable.

There is thus a need to find cosmetic compositions, especially hair compositions, that are capable of imparting a volume effect to these fibers without having the drawbacks mentioned above.

In addition, it may also be advantageous to provide such an effect coupled with coloration that is especially resistant to shampooing, so as to have available a “two-in-one” product which makes it possible to avoid degradation of the volumizing effect during a subsequent harsh dyeing treatment.

Similarly, it is advantageous to obtain the volume effect (volumization or rigidification) by giving the hair sheen or increasing its sheen, and doing so in a natural and sufficiently intense manner, without presenting the drawbacks encountered with the known processes. These processes generally consist in using compositions comprising lubricant hydrophobic substances, such as organic oils or waxes or silicones. However, the sheen effect obtained lacks intensity, very often gives the hair an artificial appearance and has the drawback of making the hair feel greasy or sticky, which is unsatisfactory.

It has been found, entirely surprisingly and unexpectedly, that it was possible to impart a volume effect to human keratin fibers, and in particular to the hair, or, in other words, to rigidify or volumize or texturize said fibers by synthesizing molecules of high molecular weight at the surface of, and/or inside, the fiber, via the polymerization of certain precursors “in situ” at the surface of, and/or inside, the fiber.

When the polymerization leads to colored molecules, the volume effect is accompanied by a coloring effect that is shampoo-resistant.

Moreover, it has been noted that the invention imparted a visual effect to the fibers treated in this way, in particular passed on or increased their sheen.

Said dye precursors are thiophenes or bisthiophenes, which, under oxidizing conditions, polymerize to form polymers according to the scheme:

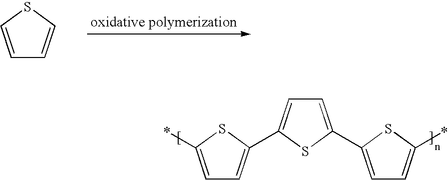

This polymerization is known and described especially by the authors Rasmussen S. C., Pickens J. C. and Hutchison J. E. in the article taken from the review Chem. Mater. 1998, Vol. 10, No. 7, pages 1990-1999—“A new, general approach to tuning the properties of functionalized polythiophenes: The oxidative polymerization of monosubstituted bithiophenes”.

A first subject of the present invention is thus the use of at least one thiophene or bisthiophene as an agent imparting a volume effect to human keratin fibers, and more particularly the hair, in a composition for treating such fibers.

A subject of the invention is also the use of at least one thiophene or bisthiophene capable of generating via oxidative polymerization a polymer that absorbs in the visible spectrum, as an oxidation dye precursor, in a composition for dyeing human keratin fibers, and more particularly the hair.

A subject of the invention is also a composition for treating or dyeing human keratin fibers, and more particularly the hair, comprising at least one thiophene or one bisthiophene in a cosmetically acceptable medium.

The invention is also directed toward a treatment process for imparting a volume and/or visual effect, such as sheen, to human keratin fibers, and more particularly the hair, which consists in applying to said wet or dry fibers a treating composition comprising at least one thiophene or one bisthiophene in a cosmetically acceptable medium, the effect being revealed using a composition comprising at least one oxidizing agent, which is mixed with the treating composition at the time of use, or which is applied sequentially without intermediate rinsing.

The invention is also directed toward a process for dyeing human keratin fibers, and more particularly the hair, which consists in applying to the wet or dry fibers a dye composition comprising, in a cosmetically acceptable medium, at least one thiophene or one bisthiophene capable of generating via oxidative polymerization a polymer that absorbs in the visible spectrum, the coloration being revealed using a composition comprising at least one oxidizing agent, which is mixed with the dye composition at the time of use, or which is applied sequentially without intermediate rinsing.

Finally, a subject of the invention is a multicompartment device or “kit” for the treatment aiming to give a volume effect, a visual effect, such as sheen, or for oxidation dyeing of human keratin fibers, and more particularly the hair, which comprises at least one compartment containing a composition comprising at least one thiophene or one bisthiophene and a second compartment containing a composition comprising at least one oxidizing agent.

The shades obtained with said molecules are moreover of satisfactory strength.

However, other characteristics and advantages of the invention will emerge more clearly on reading the description and the example that follow.

For the purposes of the present invention, the expression “volume effect, or volumization or rigidification or texturization” means a modification of the characteristics of the treated fibers, and more particularly the hair, which is reflected either by an increase in the overall volume of the fibers or by an increase in mass of the fibers, or by an improvement in their mechanical characteristics.

The evaluation of these characteristics may be performed via a sensory evaluation or via the imaging technique (volume), by means of traction tests, inter alia.

It should be noted that the optical effect covers sheen effects, but also metallic effects, goniochromatic effects, shimmering effects, fluorescent effects, thermochromic effects and electrochromic effects, inter alia.

More particularly, for the purposes of the present invention, the term “sheen” means the light intensity reflected at an angle α when the lock of hair is illuminated at an angle −α. The angle α conventionally used to measure this specular reflection, in other words the sheen, is equal to 20°. This provision of sheen may be measured using a glossmeter as described, for example, in standard ISO 2813-1994 of the AFNOR (August 1994, amended February 1997).

Among the thiophenes or bisthiophenes that are preferably used according to the present invention are those of formulae (I) to (III) below:

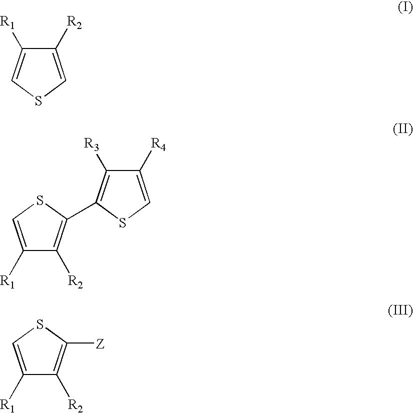

in which formulae:

R₁, R₂, R₃ and R₄, which may be identical or different, are chosen from the group formed by hydrogen, a radical —R′, —OR′, —COOR′ and —OCOR′, with R′ representing a linear or branched C₁-C₂₀ alkyl radical, a halogen atom (preferably chosen from chlorine, bromine and iodine), a nitro radical, a cyano radical, an alkylcyano radical, and solubilizing groups, it being understood that at least one radical from R₁ to R₄ denotes a solubilizing group,

Z represents —CH═CH₂ or —C≡CH.

The solubilizing groups are preferably chosen from the group formed by:

a carboxylic radical (—COOH) or carboxylate radical (—COO⁻M⁺ with M representing an alkali metal such as sodium or potassium, an alkaline-earth metal, an organic amine such as a primary, secondary or tertiary amine, an alkanolamine or an amino acid),

a sulfonic radical (—SO₃H) or sulfonate radical (—SO₃⁻M⁺, M having the same definition as above),

a primary, secondary or tertiary amine radical,

a quaternary ammonium radical such as —NR′₃⁺Z⁻ with Z=Br, Cl, (C₁-C₄)alkyl-OSO₃ and R′ alkyls, which may be identical or different, linear or branched C₁ to C₂₀ alkyls, or, in the case of two of them, may form a heterocycle with the nitrogen,

a hydroxyl radical,

a C₂-C₃ polyalkene oxide radical.

The carboxylic acid or sulfonic acid functions may or may not be neutralized with a base such as sodium hydroxide, 2-amino-2-methylpropanol, triethylamine or tributylamine.

The amine radicals may or may not be neutralized with a mineral acid, such as hydrochloric acid, or with an organic acid such as acetic acid or lactic acid, for example.

Moreover, it should be noted that said solubilizing radicals may be linked to the ring via a spacer group such as, for example, a radical —R″—, —OR″—, —OCOR″— or —COOR″— with R″ representing a linear or branched C₁-C₂₀ alkyl radical optionally comprising one or more hetero atoms, such as oxygen, for example.

Preferably, the radicals R and R₁ to R₄, which may be identical or different, are chosen from hydrogen, R′, —OR′, —OCOR′ and —COOR′ with R′ representing a C₁-C₆ linear or branched alkyl radical, and from the following neutralized or nonneutralized solubilizing groups: —COOH, —CH₂COOH, —CH₂OH, —(CH₂)₆OH, —(CH₂)₃SO₃H, —O(CH₂)₃SO₃H, —O(CH₂)₃N(CH₂CH₃)₂, —[(CH₂)₂O]CH₂CH₂OH, —[(CH₂)₂O]CH₂CH₂OCH₃ with x being a mean number of between 0 and 200.

According to a first particular embodiment, a thiophene of formula (I) is used such that the radicals R₁ and R₂ together form an ethylenedioxy ring.

Among these compounds, mention may be made more particularly of the compound sold by the company Bayer under the name Baytron M®. Baytron Me is a 3,4-ethylenedioxythiophene.

According to another particular embodiment of the invention, a thiophene of formula (I) is used in which one of the radicals is a hydrogen atom, and the other is a neutralized or nonneutralized —CH₂COOH radical. This compound has the advantage of being soluble in water.

According to a third embodiment of the invention, a bisthiophene of formula (II) is used in which R₁ or R₄ denotes a neutralized or nonneutralized radical —COOH, the other radicals representing hydrogen. This compound has the advantage of being water-soluble.

The thiophenes and bisthiophenes used in the context of the invention are compounds known to those skilled in the art.

The thiophenes or bisthiophenes are generally present in the composition in contents of at least 0.0001% by weight relative to the total weight of the composition. More particularly, the content of thiophenes or bisthiophenes represents at least 0.1% by weight relative to the weight of the composition. Preferably, the content of thiophenes and bisthiophenes represents at least 2.6% by weight relative to the weight of the composition.

In addition, the thiophenes or bisthiophenes represent not more than 50% by weight relative to the total weight of the composition and preferably not more than 10% by weight relative to the total weight of the composition.

It should be pointed out that the thiophenes and bisthiophenes make it possible to obtain a polymer in which the mean number of repeating units is at least 5 and more particularly at least 10. In addition, the mean number is not more than 10 000, advantageously not more than 1 000 and preferably not more than 700.

The cosmetically acceptable medium of the composition is preferably an aqueous medium. More particularly, it may comprise water, or a mixture comprising water and one or more cosmetically acceptable organic solvents. Among the suitable solvents, mention may be made, more particularly, of alcohols such as ethyl alcohol, isopropyl alcohol, benzyl alcohol and phenylethyl alcohol, or of glycols or glycol ethers such as, for example, ethylene glycol monomethyl, monoethyl or monobutyl ether, propylene glycol or ethers thereof such as, for example, propylene glycol monomethyl ether, butylene glycol, dipropylene glycol and also diethylene glycol alkyl ethers, for instance diethylene glycol monoethyl ether or monobutyl ether, or alternatively of polyols, for instance glycerol. Polyethylene glycols and polypropylene glycols and mixtures of all these compounds may also be used as solvent.

The concentration of the solvents, if present, may be between 0.5% and 20% by weight and preferably between 2% and 10% by weight relative to the total weight of the composition.

The dye composition may also comprise an effective amount of other agents, which are previously known elsewhere in the dyeing of human keratin fibers, such as various common adjuvants, for instance surfactants that are well known in the prior art and of anionic, cationic, nonionic, amphoteric or zwitterionic type or mixtures thereof, thickeners, antioxidants, fragrances, dispersants, conditioners especially including cationic or amphoteric polymers, opacifiers, sequestering agents such as EDTA and etidronic acid, UV-screening agents, waxes, volatile or nonvolatile, cyclic or linear or branched silicones, which are organomodified (especially with amine groups) or unmodified, preserving agents, ceramides, pseudoceramides, plant, mineral or synthetic oils, vitamins or provitamins, for instance panthenol, and nonionic, anionic, amphoteric or cationic associative polymers.

Preferably, the composition according to the invention comprises one or more surfactants that may be chosen, without preference, alone or as mixtures, from anionic, amphoteric, nonionic, zwitterionic and cationic surfactants.

The surfactants that are suitable for carrying out the present invention are especially the following:

(i) Anionic Surfactant(s):

As examples of anionic surfactants which can be used, alone or as mixtures, in the context of the present invention, mention may be made in particular (nonlimiting list) of salts (in particular alkaline salts, especially sodium salts, ammonium salts, amine salts, amino alcohol salts or magnesium salts) of the following compounds: alkyl sulfates, alkyl ether sulfates, alkylamido ether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; (C₆-C₂₄) alkyl sulfosuccinates, (C₆-C₂₄) alkyl ether sulfosuccinates, (C₆-C₂₄) alkylamide sulfosuccinates; (C₆-C₂₄) alkyl sulfoacetates; (C₆-C₂₄) acyl sarcosinates and (C₆-C₂₄) acyl glutamates. It is also possible to use the carboxylic esters of (C₆-C₂₄) alkylpolyglycosides, such as alkylglucoside citrates, alkylpolyglycoside tartrates and alkylpolyglycoside sulfosuccinates, alkylsulfosuccinamates; acyl isethionates and N-acyltaurates, the alkyl or acyl radical of all of these various compounds preferably containing from 12 to 20 carbon atoms, and the aryl radical preferably denoting a phenyl or benzyl group. Among the anionic surfactants which can also be used, mention may also be made of fatty acid salts such as the salts of oleic, ricinoleic, palmitic and stearic acids, coconut oil acid or hydrogenated coconut oil acid; acyl lactylates in which the acyl radical contains 8 to 20 carbon atoms. Alkyl-D-galactosideuronic acids and their salts, polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, in particular those containing from 2 to 50 alkylene oxide, in particular ethylene oxide, groups, and mixtures thereof can also be used.

(ii) Nonionic Surfactant(s):

The nonionic surfactants are also compounds that are well known per se (see in particular in this respect “Handbook of Surfactants” by M. R. Porter, published by Blackie & Son (Glasgow and London), 1991, pp. 116-178) and, in the context of the present invention, their nature is not a critical feature. Thus, they can be chosen in particular from (nonlimiting list) polyethoxylated or polypropoxylated alkylphenols, α-diols or alcohols having a fatty chain containing, for example, 8 to 18 carbon atoms, it being possible for the number of ethylene oxide or propylene oxide groups to range in particular from 2 to 50. Mention may also be made of copolymers of ethylene oxide and of propylene oxide, condensates of ethylene oxide and of propylene oxide with fatty alcohols; polyethoxylated fatty amides preferably having from 2 to 30 mol of ethylene oxide, polyglycerolated fatty amides containing on average 1 to 5, and in particular 1.5 to 4, glycerol groups; polyethoxylated fatty amines preferably having from 2 to 30 mol of ethylene oxide; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides.

(iii) Amphoteric or Zwitterionic Surfactant(s):

The amphoteric or zwitterionic surfactants, whose nature is not a critical feature in the context of the present invention, can be, in particular (nonlimiting list), aliphatic secondary or tertiary amine derivatives in which the aliphatic radical is a linear or branched chain containing 8 to 18 carbon atoms and containing at least one water-soluble anionic group (for example carboxylate, sulfonate, sulfate, phosphate or phosphonate); mention may also be made of (C₈-C₂₀)alkylbetaines, sulfobetaines, (C₈-C₂₀) alkylamido (C₁-C₆) alkylbetaines or (C₈-C₂₀) alkylamido (C₁-C₆) alkylsulfobetaines.

Among the amine derivatives, mention may be made of the products sold under the name Miranol, as described in U.S. Pat. Nos. 2,528,378 and 2,781,354 and classified in the CTFA dictionary, 3rd edition, 1982, under the names amphocarboxyglycinates and amphocarboxypropionates of respective structures:

R₂—CONHCH₂CH₂—N(R₃)(R₄)(CH₂COO⁻)

in which: R₂ denotes an alkyl radical of an acid R₂—COOH present in hydrolyzed coconut oil, a heptyl, nonyl or undecyl radical, R₃ denotes a β-hydroxyethyl group and R₄ denotes a carboxymethyl group; and

R₂′—CONHCH₂CH₂—N(B)(C)

in which:

B represents —CH₂CH₂OX′, C represents —(CH₂)ₙ—Y′, with

z=1 or 2,

X′ denotes a —CH₂CH₂—COOH group or a hydrogen atom,

Y′ denotes —COOH or a —CH₂—CHOH—SO₃H radical,

R₂, denotes an alkyl radical of an acid R₉—COOH present in coconut oil or in hydrolyzed linseed oil, an alkyl radical, in particular a C₇, C₉, C₁₁ or C₁₃ alkyl radical, a C₁₇ alkyl radical and its iso form, an unsaturated C₁₇ radical.

These compounds are classified in the CTFA dictionary, 5th edition, 1993, under the names disodium coco-ampho-diacetate, disodium lauro-ampho-diacetate, disodium capryl-ampho-diacetate, disodium caprylo-ampho-diacetate, disodium coco-ampho-dipropionate, disodium lauro-ampho-dipropionate, disodium capryl-ampho-dipropionate, disodium caprylo-ampho-dipropionate, lauro-ampho-dipropionic acid and coco-ampho-dipropionic acid.

By way of example, mention may be made of the cocoamphodiacetate sold under the trade name Miranol® C2M Concentrate by the company Rhodia Chimie.

(iv) Cationic Surfactants:

Among the cationic surfactants that may be mentioned in particular (nonlimiting list) are: primary, secondary or tertiary fatty amine salts, optionally polyoxyalkylenated; quaternary ammonium salts such as tetraalkylammonium, alkylamidoalkyl-trialkylammonium, trialkylbenzylammonium, trialkylhydroxyalkylammonium or alkylpyridinium chlorides or bromides; imidazoline derivatives; or amine oxides of cationic nature.

The amount of surfactants, if present in the composition according to the invention, may range from 0.01% to 40% and preferably from 0.5% to 30% of the total weight of the composition.

The oxidizing agent may be chosen from metals with redox properties, for instance iron, manganese, cobalt, copper, nickel or palladium, in free, adsorbed or complexed form, or in the form of salts, from hydrogen peroxide, urea peroxide, alkali metal bromates or ferricyanides, and persalts such as perborates and persulfates, organic oxidants such as quinones, alone or in mixtures. It is, moreover, possible to use an oxidation activator, in addition to the above-mentioned oxidizing agent. Among the activators, mention may be made of the oxygen in the air or potassium iodide.

When the oxidizing agent is hydrogen peroxide, it advantageously consists of an aqueous hydrogen peroxide solution whose titer may range more particularly from about 1 to 40 volumes and even more preferably from about 5 to 40 volumes.

Needless to say, a person skilled in the art will take care to select the optional additional compound(s) mentioned above, such that the advantageous properties intrinsically associated with the composition according to the invention are not, or are not substantially, adversely affected by the envisaged addition(s).

The composition in accordance with the invention may be in various forms, such as in the form of liquids, powders, creams or gels, which are optionally pressurized, or in any other form that is suitable for treating or dyeing human keratin fibers, and especially the hair.

The treatment process for imparting a volume effect and/or a visual effect, such as sheen, to human keratin fibers, and more particularly the hair, consists in applying to said wet or dry fibers a treating composition comprising at least one thiophene or one bisthiophene in a cosmetically acceptable medium, the volume and/or visual effect being revealed using a composition comprising at least one oxidizing agent and, where appropriate, one oxidation activator, which is mixed with the treating composition just at the time of use, or which is applied sequentially with or without intermediate rinsing.

The process for dyeing human keratin fibers, and more particularly the hair, consists in applying to said wet or dry fibers a dye composition comprising, in a cosmetically acceptable medium, at least one thiophene or one bisthiophene capable of generating via oxidative polymerization a polymer that absorbs in the visible spectrum, the coloration being revealed using a composition comprising at least one oxidizing agent and, where appropriate, one oxidation activator, which is mixed with the dye composition at the time of use, or which is applied sequentially with or without intermediate rinsing.

It should be noted that, according to a first embodiment, a composition, prepared extemporaneously just at the time of use from the treating or dyeing composition according to the invention and from the oxidizing composition, where appropriate, comprising the oxidation activator, which compositions have been described above, is applied onto the keratin fibers.

According to a second embodiment, the treatment composition or dye composition according to the invention is applied, followed by the oxidizing composition, or vice-versa; one and/or the other of these compositions optionally comprising the oxidation activator. The sequential application may be performed with or without intermediate rinsing.

Preferably, the process is performed by applying a composition obtained by extemporaneous mixing, just before use, of the treatment composition or dye composition according to the invention with the composition comprising at least one oxidizing agent, or by sequential application of the composition according to the invention and of the oxidizing composition, irrespective of the order, without intermediate rinsing.

Once the composition according to the invention and the oxidizing composition have been applied, they are left to act for an action time generally ranging from 1 to 60 minutes approximately and preferably from 10 to 45 minutes approximately.

The temperature is more particularly between 20 and 120° C., preferably between 20 and 80° C. and advantageously between 20 and 50° C.

The fibers are then optionally rinsed and are dried. It may similarly be envisioned to perform intermediate washing with shampoo and rinsing.

A concrete example illustrating the invention is given below, without, however, being limiting in nature.

### EXAMPLE

5 grams of Baytron M® from the company Bayer are dissolved in a 50/50 mixture by volume of water and ethanol. 5 g of ferric chloride are then added. Locks of natural and permanent-waved gray hair containing 90% white hairs are then immersed in this mixture, at room temperature (25° C.) (5 grams of solution per 1 gram of hair). After an action time of one hour, the locks are rinsed and are then washed with shampoo and dried.

The locks were dyed a matt green color.

The dyeing result obtained was indeed shampoo-resistant, and the hair appeared volumized (more rigid).

A comparative example without Baytron M®, performed solely with ferric chloride under the same operating conditions, colored the locks in a slightly golden shade.

1. The use of at least one thiophene or bisthiophene as an agent imparting a volume effect to human keratin fibers, in particular the hair, in a composition for treating such fibers.

2. The use of at least one thiophene or bisthiophene capable of generating via oxidative polymerization a polymer that absorbs in the visible spectrum, as an oxidation dye precursor, in a composition for dyeing human keratin fibers, and more particularly the hair.

3. The use of at least one thiophene or bisthiophene as an agent imparting a visual effect to human keratin fibers, in particular the hair, in a composition for treating such fibers.

4. The use as claimed in any one of claims 1 to 3, characterized in that the thiophenes and bisthiophenes are chosen from the group formed by those of formulae (I) to (III) below: 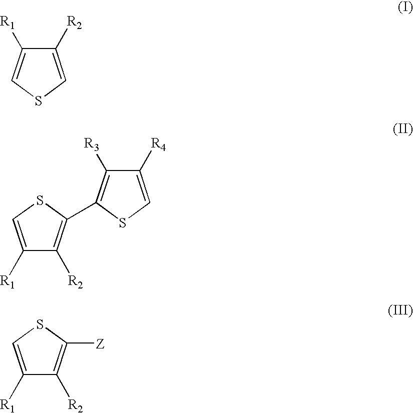
in which formulae:
R₁, R₂, R₃ and R₄, which may be identical or different, are chosen from the group formed by hydrogen, a radical —R′, —OR′, —COOR′ and —OCOR′, with R′ representing a linear or branched C₁-C₂₀ alkyl radical, a halogen atom, a nitro radical, a cyano radical, an alkylcyano radical, and solubilizing groups, it being understood that at least one radical from R₁ to R₄ denotes a solubilizing group,
Z represents —CH═CH₂ or —C≡CH.

5. The use as claimed in claim 4, characterized in that the solubilizing group(s) is(are) chosen from the group formed by:
a carboxylic radical (—COOH) or carboxylate radical (—COO⁻M⁺ with M representing an alkali metal, an alkaline-earth metal, an organic amine, an alkanolamine or an amino acid),
a sulfonic radical (—SO₃H) or sulfonate radical (—SO₃⁻M⁺, M having the same definition as above),
a primary, secondary or tertiary amine radical,
a quaternary ammonium radical such as —NR′₃⁺Z⁻ with Z=Br, Cl, (C₁-C₄)alkyl-OSO₃ and R′ alkyls, which may be identical or different, linear or branched C₁ to C₂₀ alkyls, or, in the case of two of them, may form a heterocycle with the nitrogen,
a hydroxyl radical,
a C₂-C₃ polyalkene oxide radical.

6. The use as claimed in either of claims 4 and 5, characterized in that, in formula (I), R₁ and R₂ together form an ethylenedioxy ring.

7. The use as claimed in either of claims 4 and 5, characterized in that, in formula (I), one of the radicals is a hydrogen atom and the other is a neutralized or nonneutralized —CH₂COOH radical.

8. The use as claimed in either of claims 4 and 5, characterized in that, in formula (II), R₁ or R₄ denotes a —COOH radical which has been neutralized or otherwise, the other radicals representing hydrogen.

9. A composition for treating or dyeing human keratin fibers, and more particularly the hair, characterized in that it comprises at least one thiophene or bisthiophene of formulae (I) to (III) below: 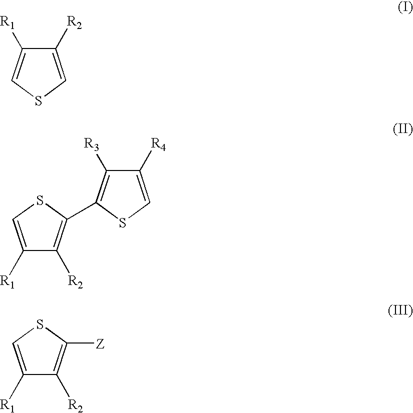
in which formulae:
R₁, R₂, R₃ and R₄, which may be identical or different, are chosen from the group formed by hydrogen, a radical —R′, —OR′, —COOR′ and —OCOR′, with R′ representing a linear or branched C₁-C₂₀ alkyl radical, a halogen atom, a nitro radical, a cyano radical, an alkylcyano radical, and solubilizing groups, it being understood that at least one radical from R₁ to R₄ denotes a solubilizing group,
Z represents —CH═CH₂ or —C≡CH,
in a cosmetically acceptable medium.

10. The composition as claimed in the preceding claim, characterized in that the solubilizing group(s) is(are) chosen from the group formed by:
a carboxylic radical (—COOH) or carboxylate radical (—COO⁻M⁺ with M representing an alkali metal, an alkaline-earth metal, an organic amine, an alkanolamine or an amino acid),
a sulfonic radical (—SO₃H) or sulfonate radical (—SO₃⁻M⁺, M having the same definition as above),
a primary, secondary or tertiary amine radical,
a quaternary ammonium radical such as —NR′₃⁺Z⁻ with Z=Br, Cl, (C₁-C₄)alkyl-OSO₃ and R′ alkyls, which may be identical or different, linear or branched C₁ to C₂₀ alkyls, or, in the case of two of them, may form a heterocycle with the nitrogen,
a hydroxyl radical,
a C₂-C₃ polyalkene oxide radical.

11. The composition as claimed in either of claims 9 and 10, characterized in that the thiophene(s) or bisthiophene(s) is(are) present in an amount of at least 0.0001% by weight, more particularly of at least 0.1% by weight, preferably of at least 2.6% by weight, relative to the total weight of the composition.

12. The composition as claimed in one of claims 9 to 11, characterized in that the thiophene(s) or bisthiophene(s) is(are) present in an amount of at most 50% by weight, more particularly of at most 10% by weight, relative to the total weight of the composition.

13. The composition as claimed in any one of claims 8 to 11, characterized in that the cosmetically acceptable medium is chosen from the group formed by water and a water-solvent mixture.

14. The composition as claimed in claim 13, characterized in that the solvent(s) is(are) chosen from alcohols, glycols, glycol ethers, polyols, polyethylene glycols and polypropylene glycols, and mixtures thereof.

15. The composition as claimed in any one of claims 8 to 14, characterized in that it comprises at least one nonionic, anionic, cationic, amphoteric or zwitterionic surfactant.

16. A treatment process for imparting a volume effect and/or a visual effect to human keratin fibers, and more particularly the hair, which consists in applying to said fibers, wet or dry, a treating composition comprising at least one thiophene or one bisthiophene in a cosmetically acceptable medium, the volume effect being revealed using a composition comprising at least one oxidizing agent and, where appropriate, one oxidation activator, which is mixed with the treating composition at the time of use, or which is applied sequentially with or without intermediate rinsing.

17. A process for dyeing human keratin fibers, and more particularly the hair, which consists in applying to said fibers, wet or dry, a dye composition comprising, in a cosmetically acceptable medium, at least one thiophene or one bisthiophene capable of generating via oxidative polymerization a polymer that absorbs in the visible spectrum, the coloration being revealed using a composition comprising at least one oxidizing agent and, where appropriate, one oxidation activator, which is mixed with the treating composition just at the time of use, or which is applied sequentially with or without intermediate rinsing.

18. The treatment process as claimed in either of claims 16 and 17, characterized in that the thiophenes or bisthiophenes are chosen from those of formulae (I) to (III) below: 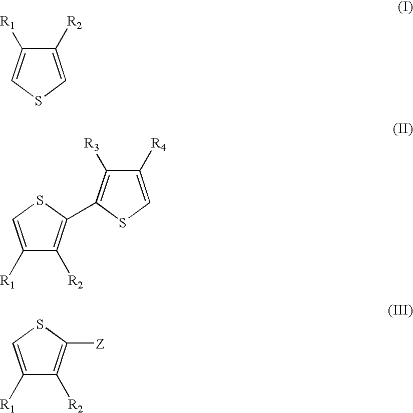
in which formulae:
R₁, R₂, R₃ and. R₄, which may be identical or different, are chosen from the group formed by hydrogen, a radical —R′, —OR′, —COOR′ and —OCOR′, with R′ representing a linear or branched C₁-C₂₀ alkyl radical, a halogen atom, a nitro radical, a cyano radical, an alkylcyano radical, and solubilizing groups, it being understood that at least one radical from R₁ to R₄ denotes a solubilizing group,
Z represents —CH═CH₂ or —C≡CH.

19. The process as claimed in the preceding claim, characterized in that the solubilizing group(s) is(are) chosen from the group formed by:
a carboxylic radical (—COOH) or carboxylate radical (—COO⁻M⁺ with M representing an alkali metal, an alkaline-earth metal, an organic amine, an alkanolamine or an amino acid),
a sulfonic radical (—SO₃H) or sulfonate radical (—SO₃⁻M⁺, M having the same definition as above),
a primary, secondary or tertiary amine radical,
a quaternary ammonium radical such as —NR′₃⁺Z⁻ with Z=Br, Cl, (C₁-C₄)alkyl-OSO₃ and R′ alkyls, which may be identical or different, linear or branched C₁ to C₂₀ alkyls, or, in the case of two of them, may form a heterocycle with the nitrogen,
a hydroxyl radical,
a C₂-C₃ polyalkene oxide radical.

20. The process as claimed in any one of claims 15 to 19, characterized in that the oxidizing agent is chosen from the group formed by redox metals in free, adsorbed or complexed form, peroxides, alkali metal bromates and ferricyanides, and persalts.

21. A multicompartment device for the treatment aiming to impart a volume effect, visual effect, or for oxidation dyeing of human keratin fibers, and more particularly the hair, which comprises at least one compartment containing a composition comprising at least one thiophene or one bisthiophene and a second compartment containing a composition comprising at least one oxidizing agent.
